(19) United States
(12) Patent Application Publication
(10) Pub. No.: US 2010/0145129 A1
(43) Pub. Date: Jun. 10, 2010

(54) EXTRACTION METHODS AND APPARATUS

(75) Inventors: Mansour Ali AL-SUGAIR, Riyadh (SA); Khaled SEAFAN, Riyadh (SA)

Correspondence Address: BUCHANAN, INGERSOLL & ROONEY PC, POST OFFICE BOX 1404, ALEXANDRIA, VA, 22313-1404, US

(73) Assignee: Awazel International Company W.L.L., Manama (BH)

(21) Appl. No.: 12/629,318

(22) Filed: Dec. 2, 2009

Related U.S. Application Data
(63) Continuation of application No. 10/518,648, filed on Dec. 2, 2005, now abandoned.

(30) Foreign Application Priority Data
Jun. 18, 2002 (GB) 0213948.3
Jun. 18, 2002 (GB) 0213949.1
Jun. 28, 2002 (GB) 0214940.9

(51) Int. Cl. C07C 7/10; B08B 5/04; B08B 3/08; C10G 17/00; C10G 1/04; C10G 29/00; A47L 9/00

(52) U.S. Cl. 585/833; 134/21; 134/19; 208/13; 208/390; 208/298; 15/300.1

(57) ABSTRACT

The present invention relates to hydrocarbon extraction method and apparatus therefor.

The present invention relates to extraction methods and apparatus therefor.

According to one aspect of the present invention a method and apparatus for cleaning a vessel, which includes sludge ponds, contaminated with a hydrocarbon is provided.

Industry is reliant upon oil and its derivatives for all manner of products, uses etc. As a consequence there are vast numbers of oil producing and processing plants around the world. These plants comprise a vast number of storage tanks for crude oil or petroleum products and sludge ponds which over a period of time accumulate large amounts of sludge and therefore require cleaning from time to time to enable corrective maintenance and inspection under preventative maintenance programmes. Sludge ponds, also known as land pits, also pose a substantial environmental hazard which has to be addressed.

As a consequence, heavy sludge deposits in such tanks and ponds must be removed. These deposits can be up to several metres thick at least in part.

Existing techniques known for cleaning such contaminated vessels consist of removing such deposits using manual labour, using eg. shovels and buckets, sometimes with the aid of water under pressure. Diesel may also be used as an inexpensive softening agent.

This technique of cleaning is labour intensive, inefficient, and often results in large quantities of contaminated waste water.

It would therefore be advantageous to overcome or alleviate one or more of the problems associated with the prior art.

In accordance with a first aspect of the present invention there is provided a method for cleaning a vessel contaminated with a sludge comprising the steps of:—

i) generating a vacuum in a conduit.

ii) contacting said sludge with said conduit;

iii) extracting said sludge via said conduit; and

iv) collecting the sludge.

Sludge typically comprises settled solids of hydrocarbons, such as asphaltenes and waxes, and inorganic solids such as sand, scale or barite. Sludge can comprise water. If asphaltenes are present with water, then water tends to be entrained in the asphaltenes which makes treatment extremely difficult by methods known in the art.

Preferably the vacuum is generated pneumatically.

Preferably the vacuum is generated by a pump.

The sludge may be heated before and/or during extraction.

The sludge may be softened before and/or during extraction with a solvent.

Such solvents comprise citrus oil extract, preferably orange oil.

In accordance with a second aspect of the present invention there is provided an apparatus for cleaning a vessel contaminated with a sludge, comprising means for generating a vacuum, a conduit connected to said means for generating a vacuum adapted to extend into the interior of the vessel from said means for generating a vacuum, and to extract the hydrocarbon and means for collecting the extracted hydrocarbon.

Preferably the means for generating a vacuum is pneumatic.

Preferably the means for generating a vacuum is a pump

The apparatus may comprise heating means for heating the hydrocarbon before and/or during extraction. This heating means can be created by oil, or electricity, or steam, or gas, or microwave. The heat is directed at the area to be cleaned with or without a fan, to the hydrocarbon as a whole or by section as appropriate. Preferably the heating means comprises a microwave emitter or heated oil.

Preferably this apparatus includes a suction head attached to said conduit. Preferably the heating means is bent into said head.

As an alternative to heating means in the apparatus itself, the receptacle in which the sludge is located may include heating means which may be used to heat the sludge.

The hydrocarbon is preferably heated to a temperature of 40-90° C., more preferably to a temperature of 65-75° C.

The apparatus is preferably portable.

Means for generating a vacuum, e.g. a suction pump may be operated by diesel, electricity or air, but preferably air.

The sludge extracted by the vacuuming action is directly transferred to receptacles such as, but not exclusively, drums, skips or vacuum tankers. Preferably, the sludge is transferred directly into a vacuum tanker.

The types of sludge that can be removed includes but are not limited to, the following list detailed with the acceptable viscosity ranges:

Asphalt (1400 to 2000 cps, but preferably 1750 to 1850 cps)

Bitumen (1000 to 1500 cps but preferably 1150 to 1250 cps)

Crude Oil (400 to 850 cps, but preferably 600 to 700 cps)

Heavy Fuel Oil.

Benefits which may result from use of the present invention include
(a) speed of sludge removal
(b) increased health and safety
(c) avoidance of the conventional multiple handling of sludge, from shovels, to buckets, to larger containers and eventually to a sludge pond.
(d) avoidance of adverse environmental impact.

According to another aspect of the present invention a method and apparatus for extracting usable hydrocarbon material from waste products is provided.

Presently sludges comprising settled solids of hydrocarbons, such as asphaltenes and waxes, and inorganic solids such as sand, scale or barite, which have been removed from oil tanks and other like storage facilities are dumped on sites such as waste land or in land pits. Such sites present major management and environmental problems owing to the high oil content of the sludges. Such sludges are toxic and hazardous to health. In countries which operate high environmental standards, for example member countries of the European Union and the USA, landfill is not a viable option. Consequently, such sludges are held in storage until a suitable permanent disposal method can be found.

Such landfill sites are in excess of 100 m square and 4 m deep. In developed economies there are stringent restrictions on how and where waste can be processed, especially when classified as hazardous to health.

In less developed countries, such as those of the Middle East, where a more relaxed view is taken of environmental issues, this waste is spread over a land mass to allow weathering, i.e evaporation and washing away, to take place without regard to the consequences. In the Middle East this process is referred to as land farming.

In any event such sludges present a major management and environmental problem.

These sludges may however contain hydrocarbons that are useful, for example they may contain hydrocarbons, that are suitable for conversion and/or processing into bituminous material, which have long been known as a suitable material for use in the formation of surfaces (e.g. the surface layers or roadbase of flexible pavement or road structures) for city streets, highways, airfields and other construction applications as well as a water-repellent barrier for use in e.g. buildings.

It would be useful, for example to convert sludges to bitumen. Such bitumen is a finite source obtained from naturally occurring sources or pyrolytically obtained from natural oil. It has been well documented that within the next century it is believed that our natural oil reserves will expire.

It would therefore be advantageous to provide an alternative source of hydrocarbon materials e.g. bitumen, and at the same time alleviate problems associated with sludge disposal.

In accordance with a further aspect of the present invention there is provided a method for extracting hydrocarbons from waste material comprising the steps of:

i) identifying waste material with an economically valuable or environmentally hazardous concentration of hydrocarbons;

ii) treating the waste material to render the hydrocarbons more susceptible to extraction;

iii) extracting the hydrocarbons from the waste material;

iv) optionally further processing the extracted hydrocarbons into a usable product.

The concentration of hydrocarbons within the waste material is preferably more than 20% hydrocarbon oil by volume.

The treatment of the waste material to render the hydrocarbons more susceptible to extraction may be performed by a number of methods. Preferably, the treatment means is by the use of heat and/or solvents.

In accordance with a further aspect of the present invention there is provided a method for extracting hydrocarbons from solid waste material comprising the steps of:

i) mixing the solid waste material with a solvent;

ii) extracting the majority of the hydrocarbons from the mixture;

iii) heating the remaining waste hydrocarbons in the mixture to a temperature of at least 40° C.;

iv) extracting the remaining waste hydrocarbons;

v) optionally further processing the extracted hydrocarbons into a usable product.

In accordance with another aspect of the present invention there is provided a method for extracting hydrocarbons from sludge comprising the steps of:

i) heating the waste hydrocarbons to a temperature of at least 40° C.;

ii) extracting the majority of the hydrocarbons;

iii) mixing the remaining waste hydrocarbons with a solvent;

iv) extracting the remaining waste hydrocarbons;

v) optionally further processing the extracted hydrocarbons into a usable product.

In accordance with further aspect of the present invention there is provided a method for extracting hydrocarbons from sludge comprising the steps of:

i) identifying sludge comprising more than 20% hydrocarbons by volume;

ii) heating said waste hydrocarbons to a temperature of at least 40° C.;

iii) extracting said heated waste hydrocarbons;

iv) optionally further processing the extracted hydrocarbons into a usable product.

A variety of methods used to identify sludge comprising more than 20% hydrocarbons by volume may be employed, for example solvent extraction, heat extraction, gas chromatography, mass spectrometry and infra red spectrometry may be used.

The present invention allows the sludge to be collected for use as a raw material. This can then be processed as fluxent oil to be mixed with bitumen during the oxidisation process into bitumen and the like.

The sludge used may contain contaminants e.g. aggregate.

Preferably, the hydrocarbons are heated to a temperature of 40-90° C. More preferably, the hydrocarbons are heated to a temperature 65-75° C.

Preferably, the average percentage of hydrocarbons by volume in the sludge starting material is at least 50%.

In accordance with a further aspect of the present invention there is provided an apparatus for extracting recyclable hydrocarbons from waste hydrocarbons contaminated with aggregate comprising:

means for heating said hydrocarbons to a temperature of at least 40° C. and means for extracting and/or transferring said heated waste hydrocarbon to a storage means.

Preferably the waste hydrocarbons are heated to a temperature in the range of 40- to 90° C., more preferably, to a temperature on the range 65-75° C.

The means for heating may comprise a coil or a bank of tubes having a circulating liquid of thermal oil/vapour/gas or electric elements. Preferably the heating means comprise oil filled tubes.

Preferably, the hydrocarbon is heated to a depth of between 20 cm-60 cm from the upper surface of the sludge, more preferably, 40-45 cm.

The heater may be directed by immersing the heater in the sludge or directing the heat by blowers or by other directional device. Preferably, the heater is immersed in the sludge. The heater may also be placed in an area or a discrete part of the sludge to enable localised hydrocarbon extraction.

The method may be used on waste material such as asphalt, bitumen, heavy fuel oil, crude oil, animal fats, vegetable oil. Preferably, it is used on bitumen.

In accordance with yet a further aspect of the present invention there is provided a method for extracting hydrocarbons from solid waste material comprising the steps of:

i) mixing the solid waste material with a solvent;

ii) extracting the hydrocarbons from the mixture;

iii) optionally further processing the extracted hydrocarbons into a usable product.

The solid waste material may comprise lumps of bitumen, asphaltenes and compacted oily sand.

The solvent may comprise of one or more solvents selected from an aqueous solvent, a non-aqueous solvent or water.

Preferably, the steps of contacting the solid material with water and mixing with a solvent is performed in a treatment area, which may comprise an excavation lined with an impermeable barrier. Such impermeable barriers may be constructed out of concrete or polythene.

The solvent used in extracting the hydrocarbons from the solid waste material is preferably an orange oil derivative. Other solvents such as aliphatic hydrocarbon, aromatic hydrocarbon and chlorinated solvents, may also be used.

Preferably, the mixing of the solid material, water and solvent is by means of a trommel; a rotating drum, an Archimedes screw, a paddle mixer, a spray bar over moving conveyor, a screen or an attrition scrubber. Those skilled in the art will realise that the exact device used will be dictated by the volume of the waste matter being treated.

The means by which the hydrocarbons may be separated from the water may comprise a rotating disc or floating head skimmer.

According to another aspect of the present invention relates to a filtration apparatus and a method of filtration is provided.

Many processing plants give rise to exhaust gas streams which contain liquids and solid particulate material which enter and pollute the atmosphere. Such harmful substances can fall out of suspension as the gases disperse or else condense with water droplets and fall as rain.

Stringent environmental policies in certain parts of the world encourage the use of filters or scrubbers to clean up the emissions from chimneys and other sources. These devices are inefficient, dependent upon chemical activity and require frequent maintenance to ensure that they are functioning correctly.

In accordance with a further aspect of the present invention there is provided an apparatus for filtering air said apparatus comprising a plurality of chambers which communicate with one another in series, such that air can pass from one chamber to another, each of which comprises means for generating a vortex.

This invention involves the use of a series of chambers through which the air to be cleaned e.g. an emission from an industrial plant passes without any external energy being introduced. The generation of the vortex displaces the air in a direction away from the centre of the chambers causing a pressure drop and consequently a cooling action.

The diameter of the chamber is such that it can effectively decelerate the speed of the volume of air entering the chamber. The height of the chamber is such that there is sufficient airspace above the level of condensation in the bottom of the chamber to prevent the condensation from being drawn upwards by the outward flow of air into the next chamber.

The means for generating the vortex may comprise at least one conical plate. Mechanical means for generating a the vortex may also be used.

The deceleration of the speed of the air causes an expansion with cooling. The continuing inflow of air causes the air to be forced between the outer rim of the conical plate and the internal side wall of the chamber. This gives rise to a vortex.

Preferably, the at least one conical plate comprises a drainage channel around the outside edge, past which the condensed solids and liquids can drain. Each plate may be perforated, solid or of slatted construction, but preferably is solid.

One or more of the chambers may be sprayed externally with a cooling fluid to provide an additional cooling action.

The solids and liquid condensates which are removed from the gas stream are all recoverable from the bases of the individual chambers whence they can be recycled or collected for disposal.

More specifically, the waste gases containing the liquids and solids destined for removal are passed either by natural flow or with fan assistance from their origin into the chamber system. Preferably this process is carried out without the use of fan assistance.

The cooling fluid may be applied through spray nozzles and may or may not be refrigerated, but preferably it is not refrigerated. The cooling fluid can drain into a reservoir whence it is recirculated over the chambers as a continuous flow.

This cooling fluid might be water, hydrocarbon solvent or liquefied gas, but preferably water.

The chambers can be constructed of mild steel, stainless steel, other metal or fibre glass or reinforced plastic, but preferably of mild steel. The steel may or not be coated internally or externally, but preferably should not be coated externally.

Unfiltered gases can be fed via pipe work of internal diameter in the range of 8 inches to 15 inches. But preferably 10 inches.

The number of chambers in the apparatus is preferably in the range of 5 to 15 more preferably 7 to 9. Preferably one or more of the first three tanks, and more preferably at least the first tank is not subject to external cooling by fluid. The remaining tanks are preferably subjected to an external spray of a fluid which will produce a cooling effect by the process of evaporation.

The height to diameter ratio of the chambers is preferably in the range of 4:1 to 6:1 more preferably 4:1, 5:1 or 6:1, and most preferably 5:1. The chambers in the apparatus may be of differing sizes or of only one size, but are preferably all of one size.

The location of the means for generating a vortex (e.g. the conical plate) within the chamber can be at the same height from the bottom in each tank or can be staggered relative to one another along a fixed gradient, such that they are linearly staggered with respect to one another.

The conical plate is preferably situated in the range of 5 cm to 35 cm from the delivery end of the inlet pipe and more preferably situated at a distance of about 25 cm. The distance between the conical plate and the delivery end of the inlet pipe forces the gas stream outwards and into the downward vortex. The gas then rises again through the centre of the chamber and when it further comes into contact with the underside of the conical plate, the gas accelerates once again around the edges of the plate to form a new upward moving vortex in the opposite direction of the incoming gas stream. This creates turbulence which in turn releases more liquid as condensate.

Condensate and solids may be collected in the base of the tank and may be removed as required through drainage cocks, pumping or other physical extraction depending on the nature of the deposit.

The exit gases from the chimney stack at the end of the process are approximately 15° C. below ambient temperature.

Where the emissions are heavily dust laden e.g. through a cold process, sufficient energy to activate the movement of the unfiltered gasses through the might require the use of a blower without the addition of heat to create sufficient air movement

The system can be used for air conditioning systems, where a simple blower would replace the need for the conventional refrigeration, thereby substantially reducing energy consumption.

The system may also be used as a filtration device for air purification e.g. in situations where there is medical need or clean room requirement, in this case the cyclone would perform the function with just an air blower.

In accordance with a further aspect of the present invention there is provided an apparatus for filtering gas comprising one or more contaminants, said apparatus comprising a plurality of chambers which communicate with one another in series, such that gas can pass from one chamber to another, at least one of the chambers comprising an inlet port, an outlet port, an internal baffle and a receiving region below the baffle for receiving contaminants, wherein said outlet port is disposed above said baffle such that gas can pass from one chamber to another whilst contaminants are retained in the receiving region of the chamber.

The baffle may be shaped to generate a vortex. Preferably, the baffle is conical in shape.

The contaminants may be solids and/or liquids. The contaminants may be derived from an industrial process, such as waste from an oxidation tower.

The apparatus may further comprise any one or more of the above mentioned features.

In accordance with a further aspect of the present invention there is provided a method of filtering air comprising the use of an apparatus as described hereinabove.

In accordance with another aspect of the present invention, there is provided a method comprising any one or more of the embodiments of the method for cleaning a vessel contaminated with a hydrocarbon, the method for extracting usable hydrocarbon material from waste products and the method of filtration described hereinabove.

In accordance with another aspect of the present invention, there is provided a system comprising any one or more of the embodiments of the apparatus for cleaning a vessel contaminated with a hydrocarbon, the apparatus for extracting usable hydrocarbon material from waste products and the filtration apparatus described hereinabove.

Specific embodiments of the present invention will now be described, by way of example only, with reference to the accompanying figures and examples, in which:—

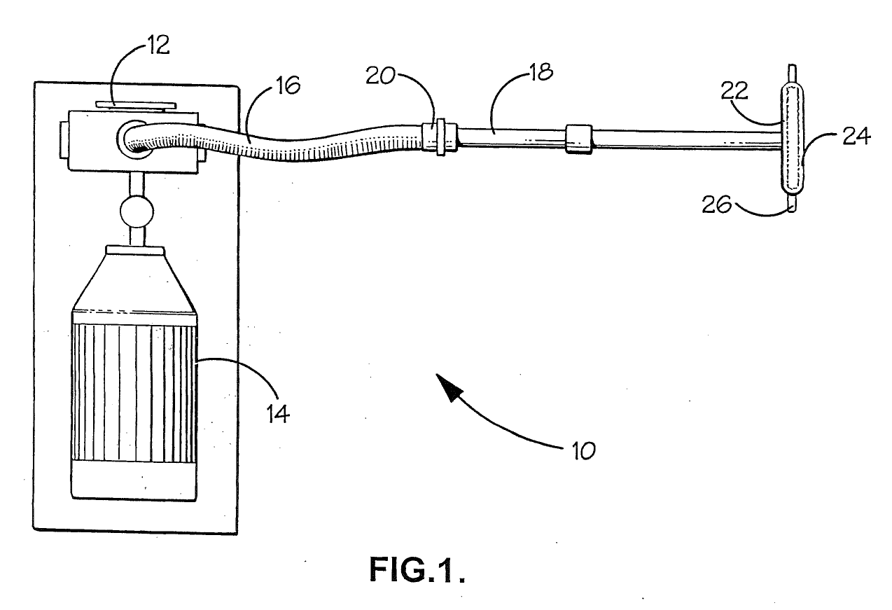

FIG. 1 is a diagrammatic view of an apparatus according to the present invention;

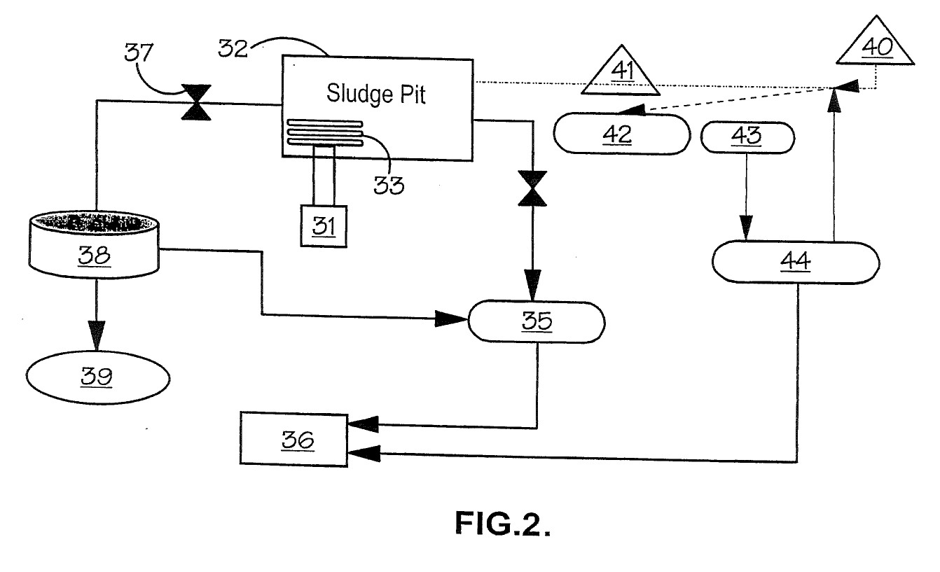

FIG. 2 is a process diagram of a method for extracting hydrocarbon material from liquid and solid phase waste; and

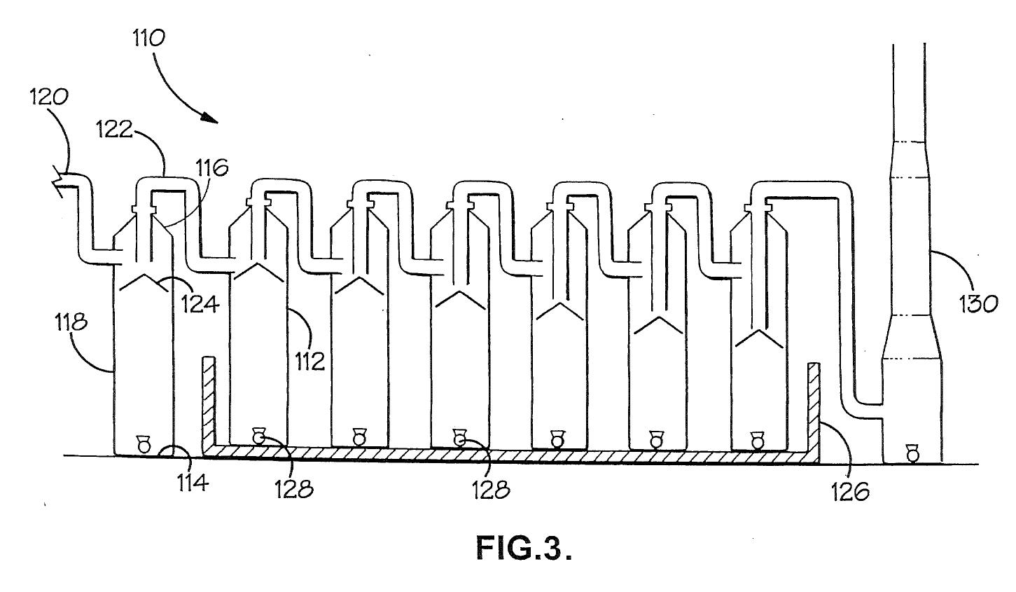

FIG. 3 illustrates an apparatus for filtering air

FIG. 1 illustrates apparatus 10 includes a pump 12 in liquid communication with a holding tank 14. Attached to pump 12 is a flexible conduit or hose 16 which is attached at its other end to an elongate, rigid hollow cylindrical member or wand 18.

Elongate member 18 has a slightly smaller diameter than hose 16 and therefor there is an adaptor 20 to ensure that the seal between the hose and elongate member 18 is airtight.

The other end of elongate member 18 communicates with an elongate head 22 whose longitudinal axis is perpendicular to that of elongate member 18. Head 22 has an opening 24 which communicates with elongate member 18. Disposed within the head 22 and adjacent to said opening is a microwave emitter 26.

During use, pump 12 is switched on to create a vacuum or suction at the opening 24 of head 22, which is placed adjacent to the hydrocarbon in a contaminated vessel whilst at the same time microwave emitter 26 softens the hydrocarbon by heating it to a temperature between 65-75° C.

Pump 12 creates a vacuum and the hydrocarbon is extracted and conveyed along the elongate member 18 and the hose 16 to the holding tank 14.

The following examples relate to FIG. 2.

### Example 1

The method for extracting hydrocarbon material from sludge in the liquid phase is as follows.

With reference to FIG. 2, the heater 31 is immersed in the sludge in a dedicated sludge pit 32. The heater in this example covers an area of 6 m² and can be a standard thermal oil heater. The heater comprises a continuously shaking coil 33, filled with thermal oil. The diameter of the coil is typically about 5 cm, although a coil in the range of 1.25 cm to 10.25 cm could also be used. The sinuosity of the coils is approximately 25 cm, but a coil with sinuosity in the range of 10 cm to 50 cm would also be acceptable. The heating temperature will depend upon the hydrocarbon concentration within the sludge.

A suction hose is laid above the heater coil, the diameter of the suction hose typically being about 10 cm in diameter. The size of the suction hose ensures that there is heat transfer into the hose, thereby keeping the sludge warm and mobile. In this regard, the suction hose may alternatively have a diameter in the range of 5 cm to 16 cm. The suction hose is connected to a displacement gear pump 34 for viscous materials which will typically be a diaphragm or a centrifugal pump.

A delivery pipe from the pump 34 feeds directly into the top of a heated tanker 35 for transport to a processing plant 36, where the recovered material is blended with hot bitumen and is then converted by standard means into waterproofing membrane or other such products or materials.

The liquid remaining in the sludge pit 32 may still contain a low quantity of hydrocarbons and can be processed further by pumping the liquid to a separation tank 38, via pump 37. The separation tank will separate water from hydrocarbons either over time or by heating and the hydrocarbons can then be moved to the heated tanker 35 for transport to a processing plant 36. The remaining water would contain a very low concentration of hydrocarbons and thus may deemed to be within a safe environmental limit. In this instance, the water may be allowed to evaporate in an evaporation pond 39.

### Example 2

The method for extracting hydrocarbon material from solid phase/material is as follows.

With reference to FIG. 2, the solid phase, which may comprise lumps of bitumen, asphaltenes and compacted oily sand 40 are skimmed off by front loader or excavator 42 down to the level where contamination levels are within acceptable levels. The materials are then transferred to a treatment area 44 by means of a dumper truck 43. A treatment area 44 may be an excavation lined with an impermeable barrier such as concrete or polythene which will be half filled with water.

The oily matter is mixed with a suitable solvent, such as a solvent with a main active ingredient being an orange oil derivative. Other solvents such as aliphatic hydrocarbon, aromatic hydrocarbon and chlorinated solvent, may also be used.

The solvent is then mixed with the oily matter. This can be achieved by a number of devices such as a trommel; a rotating drum, an Archimedes screw, a paddle mixer, a spray bar over moving conveyor, a screen, or an attrition scrubber, the exact device used is dictated largely on the volume of the waste matter being treated.

With the solvent types listed above, the oil/solvent will have a tendency to settle on top of the water. The exception is the chlorinated type where the solvent/oil will be heavier than water and this solvent may require additional processing steps which are not outlined in this example.

The waste matter containing the solvent is then pushed through the water in the treatment area by an Archimedes screw, to enable thorough mixing with the water.

Periodically, the process is halted to allow for separation of the oil from the water. At this point the free oil would be skimmed from the surface by means of a rotating disc or floating head skimmer and transferred directly to tankers for transfer to the manufacturing plant 36 for further processing (as described in example 1).

If the original material contained sand, the cleaned sand can then be excavated from the containment area and held for eventual return to the original site for further processing or disposal.

Alternatively, the cleaned sand 41 can be further cleaned by using the sludge pit 32, in the process outlined in example 1. This process can also be used if the sand 40 has a low concentration of hydrocarbons and the process of using the sludge pit 32 would be more effective than using a sand treatment area 44.

FIG. 3 is an illustration of an apparatus for filtering air.

A filtration apparatus 110 consists of seven vertically disposed, elongate cylindrical chambers 112 having a planar base 114, a conical shaped top 116 and a cylindrical side wall 118. An inlet port 120 is radially disposed in side wall 118 and an outlet port 122 in conical top 116. Outlet port 122 extends into chamber 112 and terminates near to the apex of conical plate 124. Movement of the contaminated gas over the conical plate 124 creates a vortex.

The chambers 112 are disposed in series and communicate with adjacent chambers via port 122 from one chamber which becomes inlet port 120 for the next chamber. In each successive chamber the height at which conical plates 124 are disposed relative to one another is staggered along a fixed gradient, such that they are linearly staggered.

All but the first of the chambers are disposed in a tank 126. The second to seventh chambers are subjected to external spraying with a cooling fluid to facilitate condensation of gases and thereby augment filtration. The cooling fluid is applied to the external surface of the chambers by spraying nozzles (not illustrated) and is collected in the tank 126 for recirculating. Consequently, a continuous flow of cooling fluid can be maintained.

Contaminants are collected at the bottom of the chambers 112 where extraction means 128, such as a liquid drain valve, facilitate removal of retained contaminants from the chambers 112.

The outlet port of the final chamber 112 communicates with a chimney 130 which facilitates the flow of filtered gas into the atmosphere.

1. A method for cleaning a vessel contaminated with a sludge comprising the steps of:
i) generating a vacuum in a conduit;
ii) contacting said sludge with said conduit;
iii) extracting said sludge via said conduit; and
iv) collecting the sludge.

2. A method as claimed in claim 1, wherein the vacuum is generated pneumatically.

3. A method as claimed in claim 2, wherein the vacuum is generated by a pump.

4. A method as claimed in claim 1, comprising the step of contacting the sludge with a solvent.

5. A method as claimed in claim 4, wherein the solvent comprises a citrus oil extract.

6. A method as claimed in claim 5, wherein the solvent is orange oil.

7. A method as claimed in claim 1, wherein the sludge is heated to a temperature in the range of 40-90° C.

8. A method as claimed in claim 7, wherein the sludge is heated to a temperature in the range of 65-75° C.

9. A method as claimed in claim 1, wherein the sludge comprises one or more of the group comprising asphalt, bitumen, crude oil and heavy oil.

10. An apparatus for cleaning a vessel contaminated with a sludge, comprising means for generating a vacuum, a conduit connected to said means for generating a vacuum adapted to extend into the interior of the vessel from said means for generating a vacuum, and to extract the hydrocarbon and means for collecting the extracted hydrocarbon.

11. An apparatus as claimed in claim 10, wherein the vacuum is generated pneumatically.

12. An apparatus as claimed in claim 10, further comprising means for heating the hydrocarbon before and/or during extraction.

13. An apparatus as claimed in claim 12, wherein the means for heating the hydrocarbon comprises a microwave emitter or heated oil.

14. An apparatus as claimed in claim 12, wherein said means for heating the hydrocarbon is disposed adjacent to an opening in the conduit.

15. An apparatus as claimed in claim 10, wherein the apparatus is portable.

16. (canceled)

17. A method for extracting hydrocarbons from waste material comprising the steps of:
i) identifying waste material with an economically valuable or environmentally hazardous concentration of hydrocarbons;
ii) treating the waste material to render the hydrocarbons more susceptible to extraction;
iii) extracting the hydrocarbons from the waste material;
iv) optionally further processing the extracted hydrocarbons into a usable product.

18. A method as claimed in claim 17, wherein the hydrocarbons in the waste material comprise more than 20% hydrocarbon oil by volume.

19. A method as claimed in claim 17, wherein the treatment of the waste material to render the hydrocarbons more susceptible to extraction comprises the use of heat and/or solvents.

20. A method as claimed in claim 17, wherein the extracted hydrocarbons are mixed with bitumen.

21. A method as claimed in claim 17, wherein the hydrocarbons are heated to a temperature of 40 90° C.

22. A method as claimed in claim 21, wherein the hydrocarbons are heated to a temperature of 65-95° C.

23. A method as claimed in claim 17, wherein the average percentage of hydrocarbons by volume in the waste material is at least 50%.

24. A method as claimed in claim 17, wherein the hydrocarbon is heated to a depth of between 20-60 cm from the upper surface of the sludge.

25. A method as claimed in claim 24, wherein the hydrocarbon is heated to a depth of between 40-45 cm.

26. A method as claimed in claim 17, wherein the waste material comprises one or more from the group comprising asphalt, bitumen, heavy fuel oil, crude oil, animal fats and vegetable oil.

27. A method for extracting hydrocarbons from solid waste material comprising the steps of:
i) mixing the solid waste material with a solvent;
ii) extracting the majority of the hydrocarbons from the mixture;
iii) heating the remaining waste hydrocarbons in the mixture to a temperature of at least 40° C.;
iv) extracting the remaining waste hydrocarbons;
v) optionally further processing the extracted hydrocarbons into a usable product.

28. A method for extracting hydrocarbons from sludge comprising the steps of:
i) heating the waste hydrocarbons to a temperature of at least 40° C.;
ii) extracting the majority of the hydrocarbons
iii) mixing the remaining waste hydrocarbons with a solvent;
iv) extracting the remaining waste hydrocarbons;
v) optionally further processing the extracted hydrocarbons into a usable product.

29. A method for extracting hydrocarbons from sludge comprising the steps of:
i) identifying sludge comprising more than 20% hydrocarbons by volume;
ii) heating said waste hydrocarbons to a temperature of at least 40° C.;
iii) extracting said heated waste hydrocarbons;
iv) optionally further processing the extracted hydrocarbons into a usable product.

30-32. (canceled)

33. A method for extracting hydrocarbons from solid waste material comprising the steps of:
i) mixing the solid waste material with a solvent;
ii) extracting the hydrocarbons from the mixture;
iii) optionally further processing the extracted hydrocarbons into a usable product.

34. A method as claimed in claim 33, wherein the solid waste material comprises bitumen, asphalt and compacted oily sand.

35. A method as claimed in claim 33, wherein the solvent comprises one or more selected from the group comprising aqueous solvent, non-aqueous solvent and water.

36. A method as claimed in claim 35, wherein the solvent is an orange oil derivative, aliphatic hydrocarbon, aromatic hydrocarbon or a chlarciated solvent.

37-59. (canceled)

60. A method of extraction as claimed in claim 17 comprising between steps i) and ii) a method for cleaning a vessel contaminated with a sludge comprising:
i) generating a vacuum in a conduit;
ii) contacting said sludge with said conduit;
iii) extracting said sludge via said conduit; and
iv) collecting the sludge.

61. A method of extraction as claimed in claim 1 subsequently comprising a method for extracting hydrocarbons from solid waste material comprising:
i) mixing the solid waste material with a solvent;
ii) extracting the majority of the hydrocarbons from the mixture;
iii) heating the remaining waste hydrocarbons in the mixture to a temperature of at least 40° C.;
iv) extracting the remaining waste hydrocarbons;
v) optionally further processing the extracted hydrocarbons into a usable product.

62. A method of extraction as claimed in claim 28 wherein steps i) and/or ii) comprise a method for cleaning a vessel contaminated with a sludge comprising:
i) generating a vacuum in a conduit;
ii) contacting said sludge with said conduit;
iii) extracting said sludge via said conduit; and
iv) collecting the sludge.

63. A method of extraction as claimed in claim 29 wherein steps ii) and/or iii) comprise a method for cleaning a vessel contaminated with a sludge comprising:
i) generating a vacuum in a conduit;
ii) contacting said sludge with said conduit;
iii) extracting said sludge via said conduit; and
iv) collecting the sludge.

64. A method of extraction as claimed in claim 1 subsequently comprising a method for extracting hydrocarbons from solid waste material comprising:
i) mixing the solid waste material with a solvent;
ii) extracting the hydrocarbons from the mixture;
iii) optionally further processing the extracted hydrocarbons into a usable product.

65-77. (canceled)

78. A method as claimed in claim 1 further comprising the use of an apparatus for filtering air, said apparatus comprising a plurality of chambers which communicate with one another in series, such that air can pass from one chamber to another, each of which comprises means for generating a vortex.
